(19) United States
(12) Patent Application Publication
(10) Pub. No.: US 2006/0029720 A1
(43) Pub. Date: Feb. 9, 2006

(54) Methods and apparatus for injection coating a medical device

(76) Inventors: Anastasia Panos, Worcester, MA (US); Wendy Naimark, Cambridge, MA (US); Maria Palasis, Wellesley, MA (US); Toby Freyman, Watertown, MA (US); Samuel J. Epstein, Watertown, MA (US)

Correspondence Address: KENYON & KENYON, 1500 K STREET NW, SUITE 700, WASHINGTON, DC, 20005, US

(21) Appl. No.: 10/909,408

(22) Filed: Aug. 3, 2004

(51) Int. Cl. A61L 33/00; B05C 5/00

(52) U.S. Cl. 427/2.1; 118/300

(57) ABSTRACT

Methods and apparatus for coating at least a portion of the surface of medical devices using an injection coating device are disclosed. In one embodiment, the invention includes a coating method wherein an orifice of the injection coating device is placed adjacent a vertically positioned medical device, coating material is ejected from the orifice onto the medical device, and the coating material gravitationally flows downward coating the medical device. In another embodiment, an orifice of an injection coating device is positioned adjacent a horizontally positioned medical device to gravitationally flow and deposit coating material onto the medical device. These methods may be used to apply one or more coating materials, simultaneously or in sequence. In another embodiment, multiple injection coating devices may be utilized. In certain embodiments of the invention, the coating materials include therapeutic or biologically active agents.

### FIELD OF THE INVENTION

The present invention relates to the coating of medical devices. In a specific application, the present invention relates to an apparatus and method for applying polymer coating and therapeutic materials (e.g., DNA, proteins or viruses) on the surface of an implantable medical device such as a stent.

### BACKGROUND OF THE INVENTION

The positioning and deployment of medical devices within a target site of a patient is a common, often-repeated procedure of contemporary medicine. These devices or implants are used for innumerable medical purposes including the reinforcement of recently re-enlarged lumens and the replacement of ruptured vessels.

Coatings are often applied to the surfaces of these medical devices to increase their effectiveness. These coatings may provide a number of benefits including reducing the trauma suffered during the insertion procedure, facilitating the acceptance of the medical device into the target site, and improving the post-procedure effectiveness of the device.

Coating medical devices also provides for the localized delivery of therapeutic agents to target locations within the body, such as to treat localized disease (e.g., heart disease) or occluded body lumens. Such localized drug delivery avoids the problems of systemic drug administration, such as producing unwanted effects on parts of the body which are not to be treated, or not being able to deliver a high enough concentration of therapeutic agent to the afflicted part of the body. One way to achieve localized drug delivery is by coating, for example, expandable stents, stent grafts, balloon catheters, balloon delivery systems, and aneurism coils which directly contact the inner vessel wall, with the therapeutic agent to be locally delivered. Expandable stents are tube-like medical devices that often have a mesh-like patterned structure designed to support the inner walls of a lumen. These stents are typically positioned within a lumen and, then, expanded to provide internal support for it. Because of the direct contact of the stent with the inner walls of the lumen, stents have been coated with various compounds and therapeutics to enhance their effectiveness. The coating on these medical devices may provide for controlled release, which includes long-term or sustained release, of a biologically active material.

Aside from facilitating localized drug delivery, medical devices are coated with materials to provide beneficial surface properties. For example, medical devices are often coated with radiopaque materials to allow for fluoroscopic visualization during placement in the body. It is also useful to coat certain devices to achieve enhanced biocompatibility and to improve surface properties such as lubriciousness.

Conventionally, coatings have been applied to medical devices by processes such as dipping or spraying. These coating processes are, however, inefficient, indiscriminate, wasteful, difficult to control, and/or are limited in the types of coating materials that they may apply. For example, because dip-coating or spray-coating processes often indiscriminately coat the internal surface of a patterned medical device as well as the external surface, expensive coating materials, such as therapeutic agents, are wasted, resulting in large amounts of the coating being lost during the process. Coating efficiencies of 4% are typically obtained with spraying techniques for the application of non-biologic therapeutic agents. While this may be tolerated for low cost coatings, such waste is prohibitive for expensive materials such as DNA (which may cost roughly $250 per mg), proteins or viruses. In addition, the loss of therapeutic agents into the blood stream should be minimized.

Conventional coating processes, such as dipping and spraying, also have drawbacks in the application of thick coating layers. Because thick coatings using a dip coating method require multiple dipping steps, and the dip coating solvent often dissolves a portion of the underlying dip coating upon a second dipping step, it is difficult to control the application of thick coatings. Spray coatings require multiple coating steps to achieve a desired coating thickness and do not always result in a robust coating. Thus, it is inefficient. Also, conventional spray coating processes are limited to low viscosity coating solutions and DNA and other therapeutic coating materials cannot be sprayed because the therapeutic may degrade when subjected to the high stress involved in spray methods.

In the case of stents, the indiscriminate nature of dipping may also be problematic as it may lead to the cracking and/or crumbling of coating at the junctions, hinges, and/or flexing members of the mesh-like stents. The coating that covers these portions of the stent is highly susceptible to becoming removed because, as the stent is expanded, intolerable stresses may develop within the coating. In addition, indiscriminate coating such as dip-coating and spray coating may lead to undesirable “webbing” of coating between stent members. Webbing of coating in the areas between stent members is unlikely to be held against the vessel wall, and this coating material may be lost during deployment.

The assignee of the current patent application is also the assignee of other patent applications directed to resolving some or all of the problems noted above. These include U.S. patent application Ser. No. 10/455315, filed Jun. 6, 2003, entitled “Positive Displacement Coating Deposition Apparatus and Method,” and U.S. patent application Ser. No. 10/823,636, filed Apr. 14, 2004, entitled “Methods and Apparatus for Coating a Medical Device Using a Coating Head.” The disclosures of these applications are hereby incorporated herein by reference.

Certain previously-proposed coating techniques are limited by solvent-biologically active incompatibility, low efficiencies, and coating material viscosities. There is, therefore, a need for a cost-effective method and apparatus for coating the surface of medical devices that can achieve higher weight concentrations of biologically active materials with less processing time. The method would minimize waste in coating medical devices with expensive active agents, allow for the application of coating layers of high and low viscosity, and increase the drug concentration delivery dose that can be applied using a design that is readily adaptable to high through-put manufacturing.

### SUMMARY OF THE INVENTION

The present invention regards a method and apparatus for coating at least a portion of a medical device in an efficient and effective manner.

In accordance with one embodiment, a method for applying at least a portion of a coating material on a medical device having a surface is provided. This method includes providing an injection coating device having an outlet orifice, ejecting a coating material through the outlet orifice, and allowing the coating material to flow (e.g., downwardly because of gravity) along a surface of the medical device.

In accordance with the invention, in certain embodiments, the apparatus and method are useful for applying expensive coatings, such as DNA coatings, because the apparatus and method reduce or eliminate waste of the coating material.

In accordance with the invention, in certain embodiments, the apparatus and method are useful for applying relatively viscous coatings. An apparatus and method in accordance with certain embodiments can handle highly viscous coatings, such as DNA coatings or other highly viscous coatings among those described below.

In another embodiment of the present invention, a method for applying at least a portion of a coating to a medical device having a surface is provided wherein an injection coating device ejects a second coating material through the outlet orifice and gravitationally flows the coating material along a surface of the medical device to deposit multiple layers of coating material onto the surface of the medical device.

In another embodiment of the present invention, a method for coating a series of interconnected portions of a medical device such as a stent is provided wherein a plurality of injection coating devices are used to deposit a layer of coating material, simultaneously or in stages, onto the plurality of series of interconnected portions of the medical device.

The embodiments of the injection coating system described herein provide a coating method readily adaptable to maintain a high level of automation and rapid through-put for manufacturing. They provide a facile coating system that may be used with different medical devices. They provide a cost-effective method for coating higher weight concentrations of biologically active materials with less processing time, thereby minimizing waste of expensive biologically active agents and preserving the structural integrity of the coating material.

### BRIEF DESCRIPTION OF THE DRAWINGS

FIG. 1 is an enlarged perspective view of a system for coating medical devices in accordance with a first embodiment of the present invention.

FIG. 2 is an enlarged perspective view of a system for coating medical devices in accordance with an alternative embodiment of the present invention, applying a coating to a medical device positioned horizontally.

FIG. 3 is an enlarged perspective view of a system for coating medical devices in accordance with an alternative embodiment of the present invention, incorporating a mandrel.

FIG. 4 is an enlarged perspective view of a system for coating medical devices in accordance with an alternative embodiment of the present invention, incorporating a mandrel positioned horizontally.

FIG. 5 is an enlarged side view of a system for coating medical devices in accordance with an alternative embodiment of the present invention, incorporating multiple injection coating devices that coat a medical device positioned vertically.

FIG. 6 is an enlarged side view of a system for coating medical devices in accordance with an alternative embodiment of the present invention, incorporating multiple injection coating devices that coat a medical device positioned horizontally.

FIG. 7 is an enlarged perspective view of a system for coating medical devices in accordance with an alternative embodiment of the present invention, incorporating a heating source.

FIG. 7A is an enlarged perspective view of a system for coating medical devices in accordance with an alternative embodiment of the present invention, incorporating an external heating source.

FIG. 8 is an enlarged perspective view of a system for coating medical devices in accordance with an alternative embodiment of the present invention, incorporating a cooling source.

FIG. 8A is an enlarged perspective view of a system for coating medical devices in accordance with an alternative embodiment of the present invention, incorporating an external cooling source.

FIG. 9A is an enlarged perspective view of a system for coating medical devices in accordance with an alternative embodiment of the present invention, incorporating a mandrel and an injection coating device adapted for the mandrel.

FIG. 9B is an enlarged perspective view of a system for coating medical devices in accordance with an alternative embodiment of the present invention showing a mandrel positioned within an injection coating device.

FIG. 10 is an enlarged partial side view of a system for coating medical devices in accordance with an alternative embodiment of the present invention, incorporating an injection coating device having circumferentially spaced coating delivery ports.

FIG. 11A is an enlarged bottom view taken along line 11-11 in FIG. 10 of a system for coating medical devices in accordance with an alternative embodiment of the present invention, incorporating circumferentially spaced coating delivery ports.

FIG. 11B is an enlarged bottom view taken along line 11-11 in FIG. 10 of a system for coating medical devices in accordance with an alternative embodiment of the present invention, incorporating rotating circumferentially spaced coating delivery ports.

### DETAILED DESCRIPTION

FIG. 1 illustrates a system for coating a medical device having an accessible surface in accord with the present invention. The system in this embodiment, as shown in FIG. 1 and generally designated as 10, uses an injection coating device 20 to deposit a layer of coating material 30 onto an accessible surface 41 of medical device 40. Coating material 30 can be a therapeutic agent or any liquid or paste solution.

The apparatus 20 in this embodiment comprises a piston type mechanical dispenser having a syringe barrel 21 and a syringe plunger 22. Attached at the outlet end of the syringe barrel 21 is an outlet orifice 23. The syringe plunger 22 is movable longitudinally within the syringe barrel 21. One skilled in the art can appreciate that a variety of designs may be used as injection coating device 20. For example, apparatus 20 may be a syringe, a pipette, a positive displacement deposition apparatus, or any other mechanical dispenser device known in the art. Additionally, a skilled artisan can appreciate that a micropump, a pump, an actuator, a bellows, or a bladder may be used with outlet orifice 23. Outlet orifice 23 may be a nozzle, an ultrasonic nozzle, a syringe needle, or any dispensing orifice known in the art.

As depicted in FIG. 1, the medical device 40 is positioned on a holder 50. The medical device 40 can be, for example, a stent having a patterned external surface as shown in FIG. 1. Holder 50 secures the medical device 40. The holder 50 can be, for example, a retention clip, as shown, or an inflatable balloon or mandrel, which secures the medical device by exerting a force upon the internal surface of the medical device, thereby permitting complete access to the accessible surface 41. It will be appreciated by one skilled in the art that a variety of holder devices can be designed to secure the medical device and permit access to portions of the surface of the medical device. By holding the medical device 40 from its internal surface with a holder 50 extending the length of the medical device, the holder 50 can mask the internal surface, thereby preventing the coating material 30 from adhering to the internal surface, if so desired. However, in certain applications, it may be desired to coat the interior surface of the medical device.

Referring to FIG. 1, the medical device 40, positioned on the holder 50, is then placed in close proximity to the coating head 20 to receive the coating material 30. A coating material reservoir may fluidly communicate with outlet orifice 23. The reservoir may be a separate container holding fluid outside of the syringe barrel. The separate reservoir may fluidly communicate with the syringe barrel 21 of the injection coating device 20 to replenish coating material as it is dispensed out of outlet orifice 23. Alternatively, the reservoir may be the syringe barrel interior, and fluidly communicate directly with the outlet orifice. The reservoir may contain a mixture of two or more coating materials if desired. A skilled artisan can appreciate that a variety of designs featuring the assembly of tubes, catheters, pipes and other objects can be utilized to form a fluid passageway, or facilitate the communication of coating material from a coating material reservoir to the syringe barrel 21 of the injection coating device.

Delivery of coating material is initiated by the pumping action of injection coating device 20. Pumping action is achieved by moving the syringe plunger 22 in a downward direction into the syringe barrel 21. This causes bio-compatible non-compressible coating material 30 to be pumped from the syringe barrel 21, through outlet orifice 23 of injection coating device 20, and expelled onto the medical device 40.

As illustrated in FIG. 1, the coating material 30 is deposited onto a proximal end 42 of medical device 40, which is positioned vertically and adjacent the outlet orifice 23 of injection coating device 20. The medical device 40 to be coated, for example a stent as illustrated in FIG. 1, may have struts 44 that extend from proximal end 42 to distal end 43, which are interconnected to provide support and structure to the medical device. Once deposited on proximal end 42, coating material 30 gravitationally flows downwardly towards distal end 43 along struts 44. Alternatively, medical device 40 may be placed in contact with outlet orifice 23 before coating material 30 is dispensed from orifice 23 to regulate the flow of coating material 30 and provide uniform distribution of coating.

In one embodiment, where the medical device 40 is small, for example a stent, when compared to the outlet orifice 23, neither the injection coating device 20 nor the medical device 40 need to move relative to each other to ensure full and even coating coverage of the medical device. The coating material 30, upon its release from the orifice 23, will gravitationally flow around the circumference of medical device 40.

In an alternate embodiment, where the medical device to be coated is larger than the injection coating device 20, the holder 50 may be attached to a motor, shown schematically in FIG. 1 by block 60. Holder 50 and medical device 40 are then rotated in the direction of direction arrow A as depicted in FIG. 1 to ensure a uniform layer of coating material 30 may be applied around the accessible surface 41 of medical device 40. Through rotation, the entire surface of medical device 40 to be coated can be accessed. Alternatively, portions of medical device 40 may be masked to prevent coating. The pumping action by injection coating device 20 and rotational speed of motor 60 can be controlled to allow a metered, uniform layer thickness of coating material 30 to be applied. By adjusting the width of outlet orifice 23 and controlling the rotational speed of motor 60, a thicker or thinner layer of coating material 30 can be applied. In addition, rotation may also minimize collection of coating material 30 at distal end 43.

Alternatively, holder 50 may be rotated in a direction opposite direction arrow A. In another embodiment, injection coating device 20 may be rotated in the direction of direction arrow B (as illustrated in FIG. 1), to ensure coating coverage of the proximal end 42 of medical device 40. For example, the medical device 40 shown in FIG. 1 may have the injection coating device rotated around the circumference of the proximal end 42 of the stent. Rotation of the injection coating device may be achieved by attaching a motor (not shown) to the injection coating device. In still another embodiment, both the medical device 40 and holder 50 are rotated in one direction, and the injection coating device 20 is rotated in another direction. Alternatively, medical device 40 and holder 50 may be rotated in the same direction as injection coating device 20, but at different rotational speeds.

Where the medical device 40 to be coated is substantially flat or planar, like a graft, or otherwise of an unusual shape such that rotation about its longitudinal axis will not allow application of a uniform layer of coating, the medical device 40 can be positioned vertically and translated in both the X and Y Cartesian planes under the outlet orifice 23 of the coating head to receive the layer of coating material. Alternatively, the injection coating device 20 can translate relative to the medical device 40 so that it may be able to coat the accessible surface of medical device 40. Further still, movement of both the injection coating device 20 and medical device 40 can be coordinated such that a uniform layer of coating material 30 can be applied. A skilled artisan can appreciate that medical device 40 can be masked by a variety of masking methods known in the art to prevent coating certain portions of medical device 40.

Multiple coatings may also be applied to achieve higher concentrations of coating material. For example, after the first coating material is applied and dried, a second coating material may be applied from the same injection coating device in the same manner of application as the first coating. Alternatively, a second injection coating device (not shown) may be introduced to apply the second coating material. Each coating material may be the same or a different coating solution. The properties of a multi-layer coating material may be controlled by selecting the various constituent coating materials and the order of application of the individual coating materials. For example, coatings to deliver therapeutic agents may have the therapeutic agent as the top layer coating material, or second coating material in a two-layer coating, which would be in contact with the vessel wall. A polymer binding agent may be utilized as the first coating material.

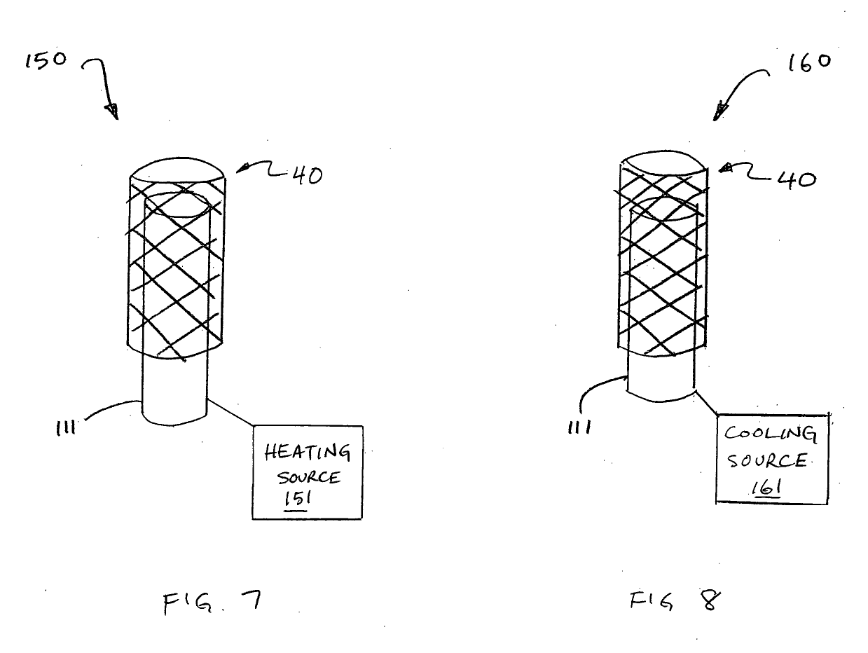

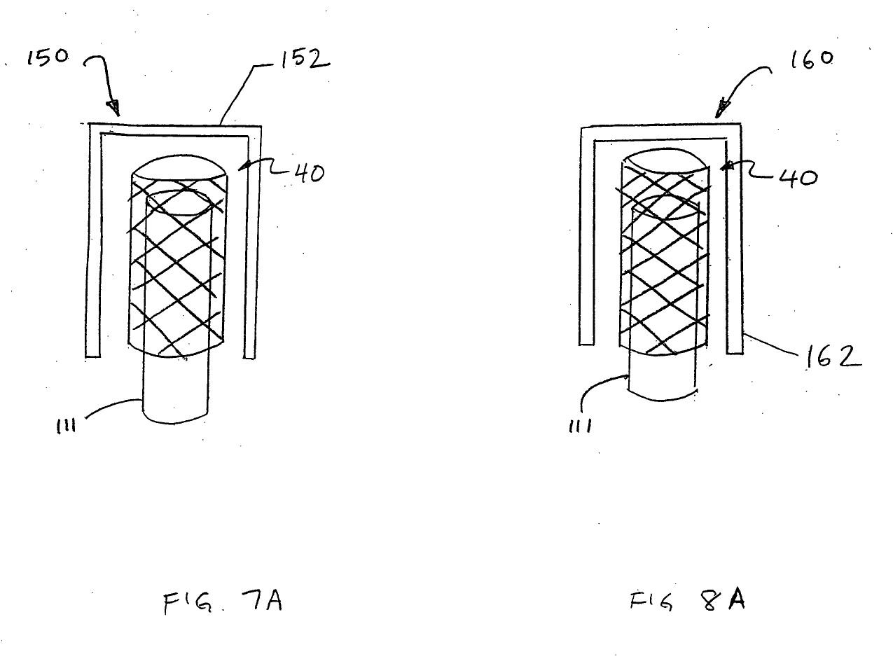

One skilled in the art can appreciate that drying may be accomplished in a variety of ways (e.g., vacuum drying) based on the coating formulation used. Alternatively, drying can be achieved by attaching a heating source to the mandrel (as illustrated in FIG. 7) or generally applying heat externally through convection, conduction, or radiation methods (as illustrated in FIGS. 7A) known in the art. In addition, the heating source may be applied to the medical device. In still another embodiment, drying may be achieved by cooling the coating material, for example, flash drying. Cooling the coating material can be accomplished by attaching a cooling source to the mandrel, as shown in FIG. 8, or to the medical device itself. Further, the cooling source may be applied externally to the medical device as illustrated in FIG. 8A.

Should it be desired to coat the medical device with a therapeutic that is hydrophobic, a hydrophilic solution may be first applied to the medical device to ensure that the hydrophobic subsequent coating flows and covers the medical device. Alternatively, the medical device may be vibrated (see FIG. 3) or rotated to permit the downward flow of the hydrophobic coating. In addition, some materials for medical devices, such as stainless steel, are hydrophobic. Thus, stainless steel medical devices generally do not have a high affinity to receive the coating solutions. Therefore, one skilled in the art can appreciate that a hydrophilic coating may be applied first to the accessible surface of the hydrophobic medical device to enhance its affinity, thereby improving the coating efficiency and increasing the available therapeutic dosage.

Pumping action of injection coating device 20 to eject coating material 30 may be achieved by a syringe (as shown in FIG. 1) or any other pumping means that can apply a pressure on the coating material 30 to dispel it from the injection coating device 20. One skilled in the art may appreciate that some of these alternative means could include a micro-pump and a collapsible bladder. In a preferred embodiment, the amount of coating material being expelled, and/or the infusion pressure placed on the coating material, will be measured to monitor the amount of coating material 30 expelled. By measuring the amount of pressure placed on the coating material the operator can monitor the progress of the procedure and thickness of the layer of the deposited coating. Thus, the coating thickness and coating flow rate can be controlled by controlling the flow rate of the coating material dispelled from the outlet orifice, and/or controlling the translation or rotational speed of the medical device to be coated.

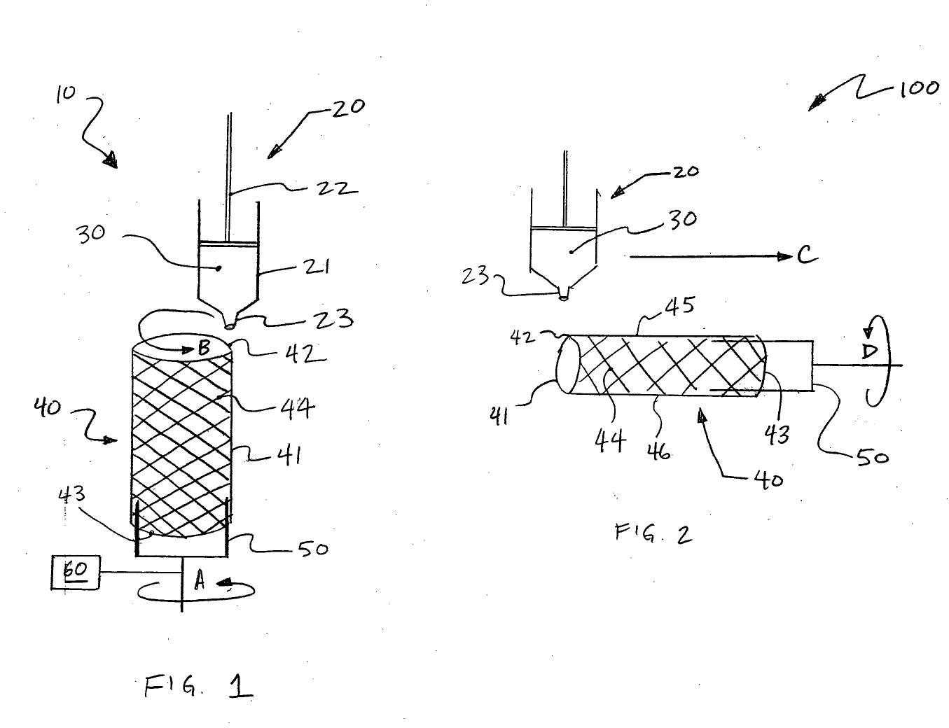

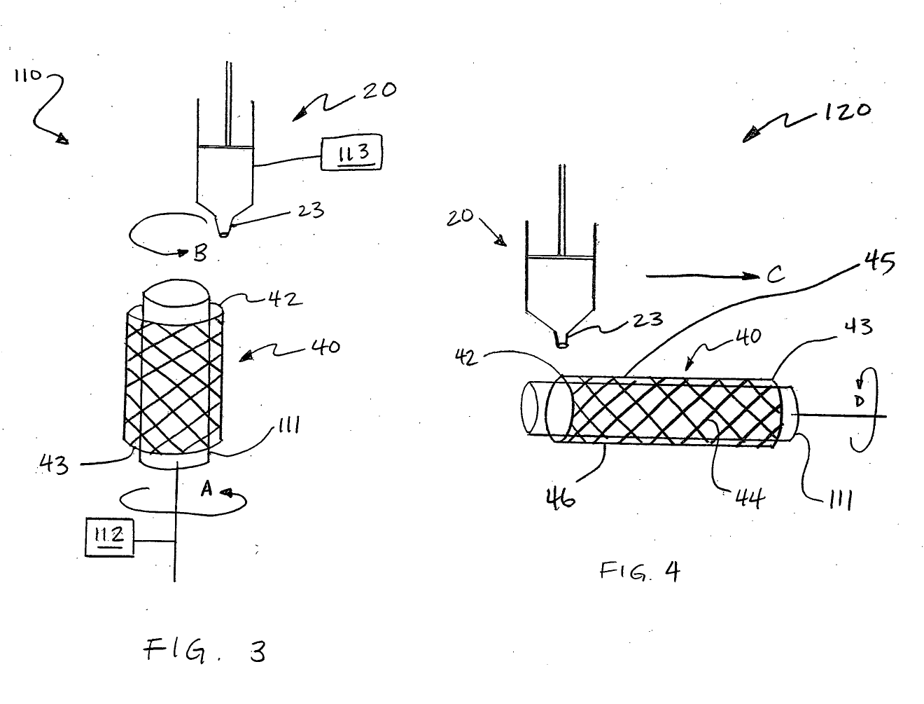

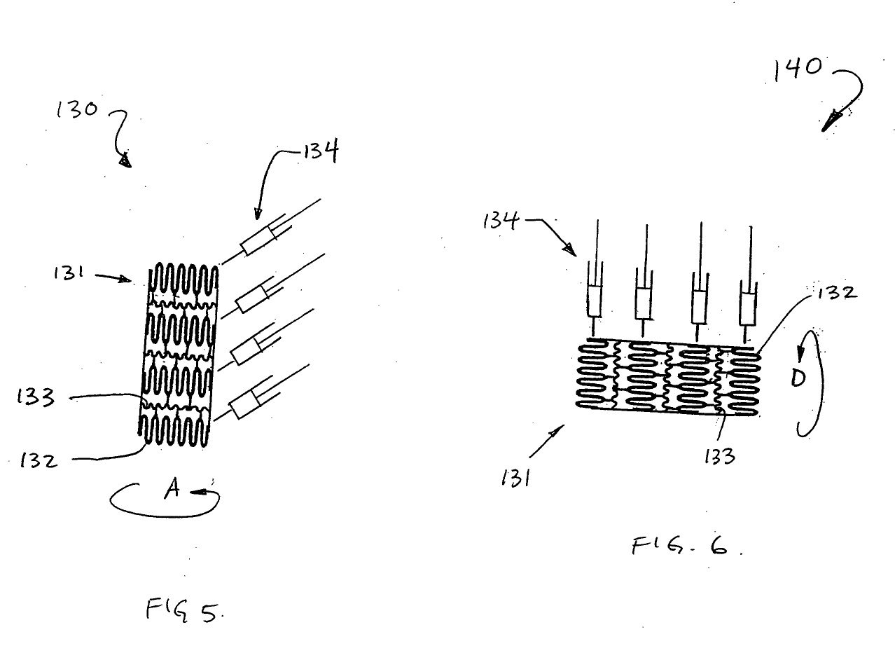

The injection coating device 20 may be made from numerous materials, including stainless steel, plastic, and other suitably rigid polymers. The holder 50, as one example, can be an inflatable balloon made with any material that is flexible and resilient. Latex, silicone, polyurethane, rubber (including styrene and isobutylene styrene), and nylon, are each examples of materials that may be used in manufacturing the inflatable balloon. Alternatively, holder 50 may be a stainless steel clip (as shown in FIGS. 1 and 2), or a bare or PTFE coated stainless steel mandrel (as shown in FIGS. 3 and 4).

In FIG. 2, an apparatus for coating a medical device in accord with another embodiment of the present invention is shown. In this embodiment, generally designated as 100, the medical device 40 is positioned horizontally with proximal end 42 and the highest part 45 of the external surface to be coated adjacent outlet orifice 23 of injection coating device 20. In this embodiment, orifice 23 is smaller than the length of the medical device 40 to be coated.

In use, coating material 30 is ejected from outlet orifice 23 onto accessible surface 41 of medical device 40 at proximal end 42. Coating material 30 gravitationally flows circumferentially around medical device 40 in a downward direction along the interconnected struts 44 from the highest part 45 to the lowest part 46 of the surface to be coated. Injection coating device 20 translates in the direction of direction arrow C from proximal end 42 towards distal end 43 of medical device 40, as shown in FIG. 2. This permits uniform coating of medical device 40.

In an alternate embodiment, where the orifice 23 is larger than the length of the medical device 40 to be coated, the medical device 40 is positioned horizontally under and adjacent outlet orifice 23. Coating material 30 is then released from outlet orifice 23 onto accessible surface 41 of medical device 40 and gravitationally flows circumferentially downward around medical device 40 along the interconnected struts 44 from the highest part 45 to the lowest part 46 of the surface to be coated.

In an alternate embodiment, holder 50 and medical device 40 may be rotated, e.g., at a constant speed, in the direction of direction arrow D as depicted in FIG. 2 to minimize collection of coating material along the lowest part 46 (the bottom edge) of medical device 40. Rotation may be achieved by attaching a motor (not shown) to holder 50. Rotating the holder and medical device also permits a uniform layer of coating material 30 to be applied around the accessible surface 41 of medical device 40. Through rotation, the entire surface of medical device 40 to be coated can be accessed. The pumping action by injection coating device 20 and rotational speed of the motor can be controlled to allow a metered, uniform layer thickness of coating material 30 to be applied. Alternatively, holder 50 and medical device 40 can also be rotated in a direction opposite direction arrow D.

In FIG. 3, an apparatus for coating a medical device in accord with another embodiment of the present invention is shown. In this embodiment, generally designated as 110, a mandrel 111 is used to secure medical device 40 and vertically position it adjacent the outlet orifice 23 of injection coating head 20 to receive the deposited coating material. The mandrel 111 can secure the medical device 40 by exerting a force upon the internal surface of the medical device. This may mask the interior surface of the medical device 40 and minimize coating of the interior. Mandrel 111 may extend beyond proximal end 42 of medical device 40, as shown in FIG. 3, to fully mask the interior and assist in positioning medical device 40. Reducing the interior lumen space of the medical device 40 forces the coating material 30 to adhere to the outer and side surfaces of the medical device 40. Preferential coating may be further optimized by adjusting the mandrel size to mask or permit coating of certain areas. Additionally, a collar (not shown) may be attached to mandrel 111 to force the coating material to flow around the collar onto proximal end 42 of the medical device 40. The collar may also be used to mask certain outer surfaces of the medical device, if desired.

The tip of the injection coating device 20 may be placed against mandrel 111 before ejecting the coating fluid for positioning and to ensure an even and consistent flow of coating material. Mandrel 111 may be a metal alloy or PTFE coated alloy. A skilled artisan may appreciate that a variety of bio-compatible materials, such as stainless steel, may be utilized. Further, the mandrel may be made of a porous material that can absorb coating material to facilitate reuse of the mandrel and avoid webbing inside the serpentine struts of certain medical devices. Alternatively, a suction source, such as a vacuum, may be used in connection with the mandrel to avoid coating material deposition on the mandrel outer surface. In another embodiment, the inner radii of the serpentine struts of certain medical devices may be masked by mandrels or spacers specially adapted to fit within the radii to preclude the formation of webbing. This would permit reuse of the mandrel and prevent coating material build-up on the mandrel surface.

A vibrating device, shown schematically as block 112 in FIG. 3, can be attached to mandrel 111 in order to shear the coating material building up and collecting longitudinally along medical device 40. Vibrating device 112 may also shear coating material beading along distal end 43 to avoid end deposition. In an alternate embodiment, vibrating device may also be attached to holder 50 in FIGS. 1 and 2. Mandrel 111 may also be rotated in the direction of direction arrow A to minimize end deposition of coating material at distal end 43, and permit a uniform layer of coating material to be applied. Alternatively, injection coating device 20 may be rotated in the direction of direction arrow B in FIG. 3.

In another embodiment, another vibrating device, shown schematically as block 113 in FIG. 3, may be attached to the injection coating device to assist in controlling the flow of coating material dispelled from orifice 23. Vibration may enhance control of coating material droplets and may allow controlled breaks in the flow of the coating material as it is dispelled from orifice 23. Vibrating or shaking the orifice may also preclude collection of coating material along the sides of orifice 23, further enhancing control over the therapeutic. Vibrating the orifice can permit the dispensing of thicker or higher viscous coating materials. One skilled in the art would appreciate that vibration can be permitted in any oscillating direction.

In FIG. 4, an apparatus for coating a medical device in accord with another embodiment of the present invention is shown. In this embodiment, generally designated as 120, the medical device 40 and mandrel 111 may be positioned horizontally with proximal end 42 adjacent outlet orifice 23 of injection coating device 20. In use, coating material is ejected from outlet orifice 23 onto medical device 40 at proximal end 42. Coating material 30 gravitationally flows circumferentially downward around medical device 40 along the interconnected struts 44 from the highest part 45 of the surface to be coated to the lowest part 46 of the surface to be coated. Mandrel 111 secures medical device 40 by applying a force to the interior surface of medical device 40, thereby masking the interior surface. Injection coating device 20 translates in the direction of direction arrow C from proximal end 42 towards distal end 43 of medical device 40, as shown in FIG. 4, allowing uniform coating of medical device 40.

Alternatively, mandrel 111 and medical device 40 may be rotated at a constant speed in the direction of direction arrow D as depicted in FIG. 4 to minimize beading of coating material along the highest part 46 of the surface to be coated of medical device 40 and allow a uniform layer of coating material to be applied. Rotation may be achieved by attaching a motor (not shown) to mandrel 111. The rotational speed of the mandrel can be controlled to allow a metered, uniform layer thickness of coating material to be applied.

In FIG. 5, an apparatus for coating a medical device in accord with another embodiment of the present invention is shown. In this embodiment, generally designated as 130, the medical device 131 may have a series of interconnected large struts 132 and small struts 133. Medical device 131 may be positioned vertically with a plurality of injection coating devices, shown generally as 134, positioned adjacent each series of large struts 132. In use, medical device 131 may be rotated in the direction of direction arrow A in FIG. 5. Coating material may then be ejected from each outlet orifice of injection coating devices 134 onto medical device 131 at the large struts 132. The coating material gravitationally flows longitudinally down medical device 131 along the interconnected struts 132 and 133. This ensures uniform coating over the entire surface and may minimize any adverse effects such as webbing (or undesired collection of coating material at the strut turns) due to large loading of the material at the top of the medical device.

In FIG. 6, an apparatus for coating a medical device in accord with another embodiment of the present invention is shown. In this embodiment, generally designated as 140, the medical device 131 may be positioned horizontally with a plurality of injection coating devices 134 positioned adjacent each series of large struts 132. In use, medical device 131 may be rotated in the direction of direction arrow D in FIG. 6. Coating material may then be ejected from each outlet orifice of injection coating devices 134 onto medical device 131 at the large struts 132. The coating material gravitationally flows circumferentially downward along the interconnected struts 132 of medical device 131. This allows an even coating and may minimize any adverse effects such as webbing due to large loading of the material at the top of the medical device. In an alternative embodiment, additional injection coating devices 134 maybe positioned adjacent the small struts 133 and utilized to ensure that the small struts are completely coated as well.

In FIG. 7, an apparatus for coating a medical device in accord with another embodiment of the present invention is shown. In this embodiment, generally designated as 150, a heating source, schematically shown as block 151, may be attached to mandrel 111 to dry the coating material. In another embodiment, a heating source may be attached to medical device 40. In still another embodiment, a heating source may be attached to a reservoir, barrel, or container holding the coating material to heat the coating material. However, if the coating material is a biologically active agent, care should be taken so as not to heat the biologically active agent above its degradation temperature. In yet another embodiment as illustrated in FIG. 7A, heat may be applied by convection or radiation from an external heating source, shown as 152, onto medical device 40.

Heat may be applied from heating source 151 or 152 to medical device 40 to facilitate the spread of high concentrations of coating material, or highly viscous coating material. Heat may also facilitate drying or phase transition gelation (to facilitate release patterns of disparate therapeutics or biologically active agents) of the coating material. One skilled in the art would appreciate that a variety of heating sources can be utilized to apply heat through convection, conduction, or radiation means.

In FIG. 8, an apparatus for coating a medical device in accord with another embodiment of the present invention is shown. In this embodiment, generally designated as 160, a cooling source, schematically shown as block 161, may be attached to mandrel 111 to cool the coating material and medical device 40. In another embodiment, a cooling source may be directly attached to medical device 40. In yet another embodiment, a cooling source may be directly attached to a reservoir, barrel, or container holding the coating material. In still another embodiment as illustrated in FIG. 8A, an external cooling source, shown as 162, may be used to cool medical device 40. Cooling medical device 40 may facilitate drying, for example flash drying, of the coating material. Cooling may also facilitate phase transition polymerization/gelation of the coating material. One skilled in the art would appreciate that a variety of cooling sources, for example a freezing probe, can be utilized to cool the medical device 40 and mandrel 111.

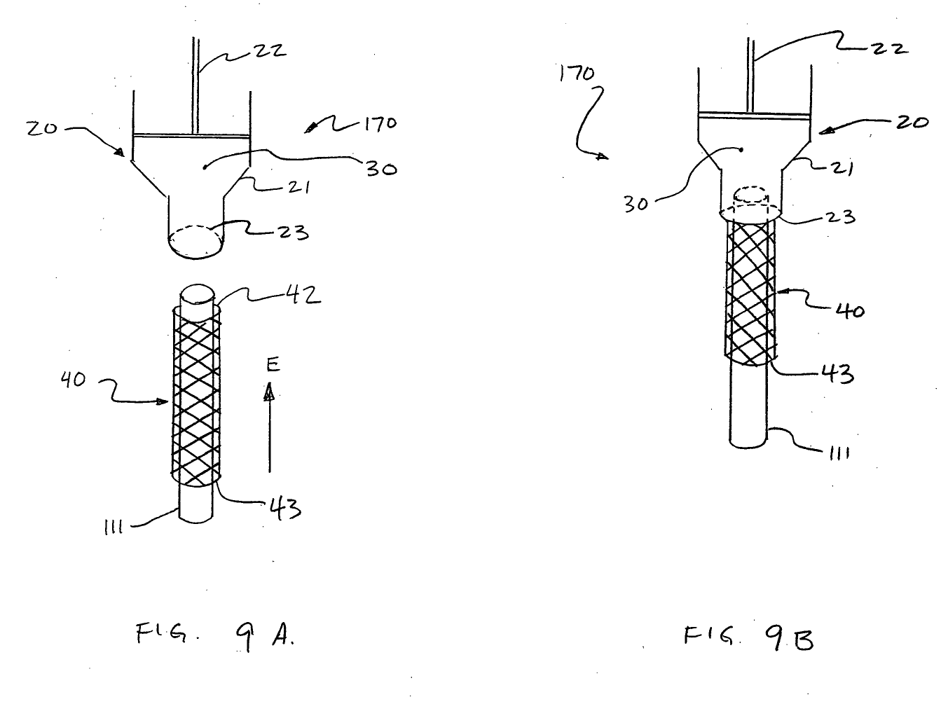

In FIG. 9A, an apparatus for coating a medical device in accord with another embodiment of the present invention is shown. In this embodiment, generally designated as 170, the medical device 40 may be positioned vertically and secured by mandrel 111. Medical device 40 and mandrel 111 are positioned adjacent outlet orifice 23 of injection coating device 20. Medical device 40 may be positioned on mandrel 111 such that the mandrel extends beyond the proximal end 42 of medical device 40, as shown in FIG. 9A.

In use, medical device 40 and mandrel 111 may be translated upward in the direction of direction arrow E in FIG. 9A towards outlet orifice 23 of injection coating device 20. Mandrel 111 may then be inserted into outlet orifice 23 of injection coating device 20, as illustrated in FIG. 9B. In this embodiment the inner diameter of the outlet orifice 23 will be slightly larger than the outer diameter of mandrel 111. One skilled in the art would appreciate that mandrel 111 may be inserted into outlet orifice 23 at a length sufficient to allow even, uniform flow (e.g., an insertion depth of 5 mm). Coating material 30 may then be ejected from the outlet orifice 23. The coating material 30 gravitationally flows downward around mandrel 111 and onto proximal end 42 of medical device 40. By depositing coating material 30 evenly around the circumference of proximal end 42, a uniform coating may be applied over the entire accessible surface of medical device 40. Coating material 30 continues flowing longitudinally down medical device 40 from proximal end 42 towards distal end 43 along the interconnected struts.

The inner diameter of the outlet orifice 23 may also be slightly larger than the outer diameter of the medical device 40. If desired, the medical device 40 may be positioned within the outlet orifice for coating.

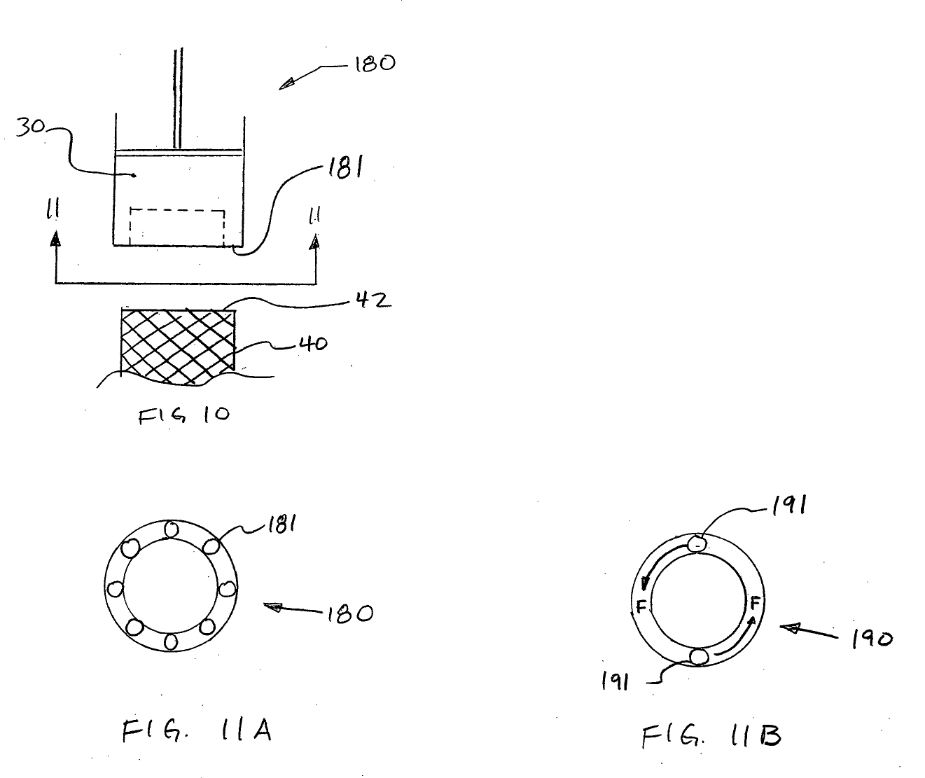

In an alternate embodiment, injection coating device 180, as generally shown in FIGS. 10, may be designed with a plurality of delivery ports 181 circumferentially spaced around the outlet orifice of the injection coating device 180, as shown in FIG. 11A which is a bottom view of FIG. 10. Injection coating device 180 may resemble, for example, a shower head design. This injection coating device 180 may be used in conjunction with any of the embodiments 10, 110, or 170 in FIGS. 1, 3, 9A, and 9B, to direct flow of coating material 30 directly onto the medical device 40. Apparatus 180 may be used with pressure augmentation, such as a syringe, or without. Apparatus 180 may be used for coating/embedding stent grafts with agents.

Another embodiment of the present invention, shown in FIG. 11B, may include an injection coating device 190 incorporating a circulating or rotating coating delivery port 191. As illustrated in FIG. 11B, which is an alternate bottom view of FIG. 10, at least one delivery port 191 may be rotated in the direction of direction arrow F to circumferentially direct the flow of coating material 30 onto the proximal end 42 of the medical device 40. In another embodiment, the injection coating device may have multiple delivery ports arranged in a linear direction (not shown) to facilitate simultaneous or staged coating of several series of struts of a medical device. Such an arrangement may be used in conjunction with any of the embodiments 130 or 140 in FIGS. 5 and 6, respectively, to direct flow of coating material directly onto the large struts 132 of medical device 131.

In another embodiment, a conveyor system (not shown) may be provided to continuously feed the medical devices towards the injection coating device. In still another embodiment, multiple medical devices could be placed into a holder, for example a round holding plate, that can accommodate many medical devices, e.g., an automatic injector system having many well plates as currently embodied in various commercialized robotic systems. Such designs would permit multiple coating steps with or without a drying step in between coatings.

Thus, the present coating system described herein discloses a coating method readily adaptable to maintain a high level of automation and rapid through-put for manufacturing. It provides a facile coating system that may be used with different medical devices and with highly complex coating combinations.

The medical devices used in conjunction with the present invention include any device amenable to the coating processes described herein. The medical device may be constructed of any biocompatible material known in the arts, for example nickel or stainless steel. The medical device, or portion of the medical device, to be coated or surface modified may be made of metal, polymers, ceramics, composites or combinations thereof. Whereas the present invention is described herein with specific reference to a vascular stent, other medical devices within the scope of the present invention include any devices which are used, at least in part, to penetrate the body of a patient. Non-limiting examples of medical devices according to the present invention include catheters, guide wires, balloons, filters (e.g., vena cava filters), stents, stent grafts, vascular grafts, intraluminal paving systems, soft tissue and hard tissue implants, such as orthopedic repair plates and rods, joint implants, tooth and jaw implants, metallic alloy ligatures, vascular access ports, artificial heart housings, heart valve struts and stents (used in support of biologic heart valves), aneurysm filling coils, and other coiled coil devices, trans myocardial revascularization (“TMR”) devices, percutaneous myocardial revascularization (“PMR”) devices, hypodermic needles, soft tissue clips, holding devices, and other types of medically useful needles and closures, and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like. Any exposed surface of these medical devices may be coated with the methods and apparatuses of the present invention.

The coating materials used in conjunction with the present invention are any desired, suitable substances. In some embodiments, the coating materials comprise therapeutic agents, applied to the medical devices alone or in combination with solvents in which the therapeutic agents are at least partially soluble or dispersible or emulsified, and/or in combination with polymeric materials as solutions, dispersions, suspensions, latices, etc. The solvents may be aqueous or non-aqueous. Coating materials with solvents may be dried or cured, with or without added external heat, after being deposited on the medical device to remove the solvent. The therapeutic agent may be any pharmaceutically acceptable agent such as a non-genetic therapeutic agent, a biomolecule, a small molecule, or cells. The coating on the medical devices may provide for controlled release, which includes long-term or sustained release, of a therapeutic agent.

Exemplary non-genetic therapeutic agents include anti-thrombogenic agents such as heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bc1-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyisobutylene copolymers and styrene-isobutylene-styrene block copolymers such as styrene-isobutylene-styrene tert-block copolymers (SIBS); polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and copolymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

In a preferred embodiment, the polymer is polyacrylic acid available as HYDROPLUS® (Boston Scientific Corporation, Natick, Mass.), and described in U.S. Pat. No. 5,091,205, the disclosure of which is incorporated by reference herein. In a more preferred embodiment, the polymer is a co-polymer of polylactic acid and polycaprolactone.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The release rate of drugs from drug matrix layers is largely controlled, for example, by variations in the polymer structure and formulation, the diffusion coefficient of the matrix, the solvent composition, the ratio of drug to polymer, potential chemical reactions and interactions between drug and polymer, the thickness of the drug adhesion layers and any barrier layers, and the process parameters, e.g., drying, etc. The coating(s) applied by the methods and apparatuses of the present invention may allow for a controlled release rate of a coating substance with the controlled release rate including both long-term and/or sustained release.

The coatings of the present invention are applied such that they result in a suitable thickness, depending on the coating material and the purpose for which the coating(s) is applied. The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers, which may perform identical or different functions. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

In addition to the previously described coating layers and their purposes, in the present invention the coating layer or layers may be applied for any of the following additional purposes or combination of the following purposes: to alter surface properties such as lubricity, contact angle, hardness, or barrier properties; to improve corrosion, humidity and/or moisture resistance; to improve fatigue, mechanical shock, vibration, and thermal cycling; to change/control composition at surface and/or produce compositionally graded coatings; to apply controlled crystalline coatings; to apply conformal pinhole free coatings; to minimize contamination; to change radiopacity; to impact bio-interactions such as tissue/blood/fluid/cell compatibility, anti-organism interactions (fungus, microbial, parasitic microorganisms), immune response (masking); to control release of incorporated therapeutic agents (agents in the base material, subsequent layers or agents applied using the above techniques or combinations thereof); or any combinations of the above using single or multiple layers.

One of skill in the art will realize that the examples described and illustrated herein are merely illustrative, as numerous other embodiments may be implemented without departing from the spirit and scope of the present invention.

What is claimed is:

1. A method for coating at least a portion of a medical device with a coating material comprising:
providing a medical device having at least one accessible surface, a proximal end, and a distal end;
holding the medical device with a holder wherein the medical device is generally vertically positioned with the proximal end above the distal end;
providing at least one injection coating device comprising an outlet orifice and a reservoir, wherein the reservoir contains a coating material and is in fluid communication with the outlet orifice;
positioning the injection coating device adjacent the proximal end of the medical device;
ejecting the coating material through the outlet orifice of the injection coating device onto the proximal end of the medical device; and
allowing the coating material to flow down at least one accessible surface of the medical device from the proximal end to the distal end, thereby depositing the coating material on at least one accessible surface of the medical device.

2. The method of claim 1 further comprising translating the medical device from a first position to a second position.

3. The method of claim 1 further comprising rotating the medical device.

4. The method of claim 1 further comprising translating the injection coating device from a first position to a second position.

5. The method of claim 1 further comprising rotating the injection coating device.

6. The method of claim 1 further comprising vibrating the holder.

7. The method of claim 1 further comprising vibrating the injection coating device.

8. The method of claim 1 further comprising drying the deposited coating material.

9. The method of claim 1 wherein the holder is a mandrel.

10. The method of claim 9 wherein the mandrel is porous.

11. The method of claim 9 wherein the mandrel comprises a vacuum core.

12. The method of claim 1 wherein the medical device is a stent.

13. The method of claim 1 wherein the injection coating device is a syringe.

14. The method of claim 1 further comprising masking at least a portion of the medical device.

15. The method of claim 1 further comprising inserting the holder into the outlet orifice of the injection coating device, wherein the ejected coating material is evenly deposited onto the proximal end of the medical device.

16. The method of claim 1 wherein the outlet orifice comprises a plurality of delivery ports circumferentially positioned along the outlet orifice.

17. The method of claim 1 wherein the outlet orifice comprises at least one delivery port wherein the delivery port is rotated circumferentially along the outlet orifice.

18. The method of claim 1 further comprising placing the medical device against the injection coating device.

19. The method of claim 1 further comprising:
ejecting at least a second coating material through the outlet orifice of the injection coating device onto the proximal end of the medical device; and
allowing the second coating material to flow down at least one accessible surface of the medical device from the proximal end to the distal end, thereby depositing the coating material on at least the first coating material forming a multi-layer coating material on the accessible surface of the medical device.

20. The method of claim 1 further comprising a plurality of injection coating devices.

21. A method for coating at least a portion of a medical device with a coating material comprising:
providing a medical device having a longitudinal axis and at least one accessible surface;
holding the medical device with a holder wherein the medical device is horizontally positioned with the longitudinal axis generally horizontally aligned;
providing an injection coating device comprising an outlet orifice and a reservoir, wherein the reservoir contains a coating material and is in fluid communication with the outlet orifice;
positioning the injection coating device above the medical device;
ejecting the coating material through the outlet orifice of the injection coating device onto the medical device; and
allowing the coating material to flow down at least one accessible surface of the medical device, thereby depositing the coating material on at least one accessible surface of the medical device.

22. The method of claim 21 further comprising translating the injection coating device from a first position to a second position.

23. The method of claim 21 further comprising rotating the medical device.

24. The method of claim 22 further comprising rotating the medical device.

25. A method for coating at least a portion of a stent with a coating material comprising:
providing a stent having at least one accessible surface, a proximal end, a distal end, and a plurality of series of interconnected struts;
holding the stent with a holder wherein the stent is generally vertically positioned with the proximal end above the distal end;
providing a plurality of injection coating devices wherein each injection coating device comprises an outlet orifice and a reservoir, wherein the reservoir contains a coating material and is in fluid communication with the outlet orifice;
positioning each injection coating device adjacent a series of interconnected struts of the stent;
ejecting the coating material through the outlet orifice of each injection coating device onto the series of interconnected struts of the stent; and
allowing the coating material to flow down at least one accessible surface of the stent from the proximal end to the distal end, thereby depositing the coating material on at least one accessible surface of the stent.

26. A method for coating at least a portion of a stent with a coating material comprising:
providing a stent having a longitudinal axis, at least one accessible surface, and a plurality of series of interconnected struts;
holding the stent with a holder wherein the stent is horizontally positioned with the longitudinal axis generally horizontally aligned;
providing a plurality of injection coating devices wherein each injection coating device comprises an outlet orifice and a reservoir, wherein the reservoir contains a coating material and is in fluid communication with the outlet orifice;
positioning each injection coating device above a series of interconnected struts of the stent;
ejecting the coating material through the outlet orifice of each injection coating device onto the series of interconnected struts of the stent; and
allowing the coating material to flow down at least one accessible surface of the stent, thereby depositing the coating material on at least one accessible surface of the stent.

27. A system for coating at least a portion of a medical device with a coating material comprising:
a holder adapted to secure a medical device; and
at least one injection coating device comprising an outlet orifice and a reservoir, wherein the reservoir contains a coating material and is in fluid communication with the outlet orifice;
wherein the holder is positioned adjacent the injection coating device to allow the medical device to receive the coating material ejected from the outlet orifice of the injection coating device.
